(19) United States
(12) Patent Application Publication
(10) Pub. No.: US 2015/0147408 A1
(43) Pub. Date: May 28, 2015

(54) METHOD VALIDATION UNIT

(71) Applicant: Australian Biotechnologies PTY. Limited, Frenchs Forest, New South Wales (AU)

(72) Inventors: Simon Jonothan Berry, Melbourne (AU); Sharon Bryce, Carlingford (AU); Jerome Alon Goldberg, Vaucluse (AU)

(73) Assignee: Austrialian Biotechnologies PTY. Limited, Frenchs Forest (AU)

(21) Appl. No.: 14/404,925

(22) PCT Filed: May 30, 2013
(86) PCT No.: PCT/AU2013/000565

§ 371 Date: Dec. 1, 2014

(30) Foreign Application Priority Data
May 31, 2012 (AU) 2012902273

(51) Int. Cl. C12Q 1/22; A61L 2/28; A61K 35/28; A61L 2/04

(52) U.S. Cl. CPC C12Q 1/22; A61L 2/04; A61L 2/28; A61K 35/28
USPC 424/549; 422/38; 435/287.4; 435/31

(57) ABSTRACT

A Method Validation Unit (MVU) for the evaluation of the level of sterilisation in a sterilisation method involving the use of a fluid at or near the supercritical pressure and temperature for that fluid, wherein the MVU comprises a sterilisation indicator housed within a gas-permeable container, wherein the sterilisation indicator comprises an indicator medium and a population of one or more colony forming units (CFUs), and wherein the indicator medium comprises one or more structural features representative of the internal structure of a material to be sterilised in the sterilisation method.

### TECHNICAL FIELD

The present invention relates to a Method Validation Unit (MVU) and particularly the method of manufacture and use of an MVU in a sterilisation method using a supercritical fluid (SCF).

### BACKGROUND OF THE INVENTION

Materials or articles used in surgery must be rigorously sterilised before use. Thorough sterilisation is required to prevent infections.

Treating such infections is inconvenient, stressful and costly. In extreme cases such infections can lead to the death of the patient. This is particularly true for vulnerable people such as the young, old, or people with compromised immune systems.

Accordingly, it is highly desirable to prevent these infections by rigorously sterilising the materials or articles to be used in surgery. This is especially true when the materials or articles are intended to remain in the body after the surgical procedure has been completed.

A number of sterilant-based sterilisation methods have been previously described in the prior art. These methods involve contacting the substrate to be sterilised with a gas or fluid sterilant under conditions suitable to bring about sterilisation. Examples of gas or fluid sterilants are steam, ethylene oxide and hydrogen peroxide. It has also been shown that SCFs (such as carbon dioxide) can be used in such sterilisation methods.

U.S. Pat. No. 6,149,864 (in the name of the Massachusetts Institute of Technology) discloses a SCF sterilisation process using supercritical carbon dioxide (SCCD). WO 2005/000364 and The Journal of Biotechnology, 2006, volume 123, issue 41, pages 504-515 (in the name of NovaSterlis Inc., hereafter the ‘Journal of Biotechnology (2006)’) discloses that the SCCD sterilisation process disclosed in U.S. Pat. No. 6,149,864 can be improved by the addition of certain additives. The subject-matter of the above-cited documents is incorporated herein by reference.

As it is difficult to determine if complete sterilisation has been affected in a substrate (without destructive testing), and because the consequences of failing to fully sterilise a substrate can be dire, it is often necessary to sterilise the substrate under very stringent conditions (overkill conditions). Overkill conditions are used to ensure that there is a vanishingly small probability that an infectious agent will survive the sterilisation process used.

For a sterilised substrate to be deemed sufficiently sterile to meet the National legal requirements for use in surgery, the substrate must be sterilised under conditions suitable to bring about a specified reduction in one or more microorganisms. The level of sterilisation brought about by a certain sterilisation process (or required of a sterilisation process) is often referred to as a Sterility Assurance Level (SAL; US FDA, 1993). The National legal requirements for sterilised articles for use in surgery will be known to the skilled person in that Nation, and these standards are incorporated herein by reference (e.g. TGA Guidelines for Sterility Testing of Therapeutic Goods: 1998; ISO 14161; ISO 14161; AS/NZS 4187—2003 and AS EN 1174.1—2002, incorporated herein by reference).

For example a SAL of 10⁻⁶ (SAL10⁻⁶) means that the sterilising conditions were sufficient to reduce the number of contaminating microorganisms to a level of 10⁻⁶ microorganisms. Clearly it is not possible for there to be a fraction (i.e. one millionth) of a live microorganism remaining in the treated substrate. Accordingly, a negative SAL value in effect represents the probability that a substrate will remain contaminated after the completion of the sterilisation process. Therefore, a SAL10⁻⁶ means that there is a one in a million chance that a substrate will remain contaminated after sterilisation. Another way to express this is to say that SAL10⁻⁶ means the sterilisation conditions would be sufficient to kill at least 10⁶ microorganisms, if these had been present in a sample undergoing that sterilization process.

The SAL of a particular sterilisation process as mentioned in the prior art cited above is determined with respect to a calibration process, the calibration process being standardised with respect to a particular bacterial or microbial species.

By running multiple samples at various time intervals a calibration graph was produced in WO 2005/000364 and in the Journal of Biotechnology (2006), which correlated run time against the log reduction in the bacterial species tested. The resulting calibration graph allows the ‘D-value’ to be determined. The D-value gives the amount of time required to cause a 10-fold reduction (i.e. 1 log unit) in the population of the bacterial species treated. The D-value in WO 2005/000364 was determined to be 14.24 minutes and 3.25 minutes in the Journal of Biotechnology (2006).

The sterilisation process described in the above-cited publications was calibrated by the use of a bacterial colony placed onto the outer surface of a glass substrate.

To maximise the effectiveness of a SCF sterilisation process, the SCF should be intimately contacted with the substrate to be sterilised. However, despite the use of various SCF circulation means, certain areas of the sterilisation chamber are exposed to the SCF sterilant to a greater degree than other areas, resulting in sterilisation ‘hotspots’ (and ‘cold spots’) in the sterilising chamber.

Calibration is also time consuming. For example, condensing the SCF into the sterilisation apparatus can lead to a twenty minute induction period before the sterilisation process can begin.

Generating a calibration graph results in the loss of valuable time, which could otherwise be used to make sterilised substrates.

The apparatus and process will also need to be periodically re-calibrated to ensure that there has been no significant divergence from the process conditions used to calibrate the apparatus and process. It is also difficult to generate exactly the same turbulent effects within the sterilising chamber during each sterilisation run, particularly when the apparatus chamber is packed differently during each sterilisation run. As such, it is possible that between each sterilisation run the locations of the ‘hot-spots’ can periodically migrate within the sterilisation chamber.

Furthermore, to have confidence in the sterilising process, the conditions used in the sterilising process should correspond closely to the sterilising conditions used in the calibration process. The resulting effect in the sterilising process of deviating from the conditions used in the calibration process cannot be reliably predicted. Any difference between the calibration and sterilisation process conditions will alter the D-value, and as such the corresponding SAL cannot be reliably estimated based on such a D-value.

In addition, despite all reasonable efforts made by an operator, there is still the possibility that an unnoticed one-off user error could occur (such as a failure to add the required level of additive), or for an unnoticed apparatus malfunction to occur. Failures of this kind could result in a substrate being sterilised under conditions which do not meet the desired or required SAL.

### SUMMARY OF THE INVENTION

According to the first aspect of the invention, there is provided a Method Validation Unit (MVU) for the evaluation of the level of sterilisation in a sterilisation method involving the use of a fluid at or near the supercritical pressure and temperature for that fluid, wherein the MVU comprises: a sterilisation indicator housed within a gas-permeable container, wherein the sterilisation indicator comprises an indicator medium and a population of one or more colony forming units (CFUs), and wherein the indicator medium comprises one or more structural features representative of the internal structure of a material to be sterilised in the sterilisation method.

In use one or more MVUs are placed in the sterilisation apparatus with the substrates to be sterilised, and the MVU and substrates are subjected to a sterilisation method employing a SCF.

The MVUs can be placed in various locations in the sterilisation chamber of the sterilisation apparatus including those areas known to be least accessible to the SCF, or the MVUs can be randomly placed in the sterilisation chamber.

After completion of the sterilising process, the sterilising chamber is returned to ambient temperature and pressure conditions (e.g. depressurised) and the one or more MVUs are removed. Each process-treated MVU can then be tested to evaluate the effectiveness of the sterilisation process used. The skilled person is aware of various ways used to evaluate the result of a sterilisation process (e.g. British Pharmacopeia/European Pharmacopeia 2012 standards, incorporated herein by reference), including incubating and counting the live microorganisms. Failure to incubate any live microorganisms would indicate that the sterilisation process was completely effective.

A MVU offers the benefit that it can be placed in the sterilisation chamber with the substrates (e.g. the materials or articles) to be sterilised. In that way the MVU is treated under exactly the same conditions as the substrates being sterilised, and so when tested the (process-treated) MVU gives a direct measure of the sterilising conditions experienced by the sterilised substrates. The MVU allows the SAL to be directly measure so that determining the SAL is not entirely reliant on an indirectly prophetic calibration process as described in the prior art.

Therefore, the use of a MVU reduces the chance that a substrate will be made and sent out (and possibly used in surgery) that has not actually been sterilised to the required (or desired) SAL. Where this SAL is below the Nationally set requirement, this is a serious matter.

The MVU also allows a one-off user error (or apparatus malfunction) to be readily identified and corrected. The MVU ensures that a user error (or apparatus malfunction) will not continue for any prolonged period of time.

The MVU allows a user error (or apparatus malfunction) to be traced to a specific batch, and so if a product recall were ever required, that product recall could be limited to only that batch. Without a MVU a wider product recall would probably be necessary, which would be costly and wasteful. Indeed, the MVU allows the batch to be directly assessed prior to the batch being sent out.

As the MVU is used to determine the effectiveness of the sterilisation process there is no need to sacrifice the sterilised substrates to quality control testing. There is a scarcity of therapeutical grade tissue which is suitable for human transplantation, so such destructive testing would is wasteful.

In any event as the substrates to be sterilised are not inoculated with a colony of microorganisms (as is the case with the MVUs), these substrates cannot be used to determine if the required (or desired) sterilisation conditions have actually been experienced by the sterilised substrates in the sterilisation process.

While it is possible to destructively test a substrate to determine if it is actually sterile, it is not possible to measure the probability of it not being sterile. For example, there is no practical way of determining if a substrate has been treated under SAL10⁻⁴ or SAL10⁻⁶ conditions by analysing the substrate alone, it is only possible to confirm that the substrate is sterile. A SAL of 10⁻⁶ means that there is a probability that one in a million sterilised substrates will not be sterile. Short of testing many sterilised substrates, there is no reasonable way of measuring this probability from the sterilised substrates themselves.

In summary, the MVU offers at least the following benefits over the prior art which is reliant on a sterilisation calibration procedure.

The MVU provides additional confidence in the SCF sterilisation process, eliminating entire reliance on the periodic calibration of the sterilisation process, particularly where it is possible for the process conditions to deviate from the conditions used in the calibration. This provides a saving on lost production time and associated costs.

The MVU allows user (and/or process) errors to be identified and quickly corrected.

The MVU can verify that the substrates formed in a sterilisation process have actually been treated under the required (or desired) sterilising conditions. The sterilised substrates benefit from the verification of this minimum SAL. The MVU gives a reduced probability that a sterilised product could be sent out that has not met the required (or desired) SAL. Therefore, a MVU reduces the probability that a defective product could be used by a medical practitioner which results in infection. Accordingly, medical practitioners would have improved confidence in using such a SAL verified product. This improved confidence could result in lower medical liability insurance.

According to a second aspect of the invention, there is provided the use of a MVU according to the first aspect of the invention in an apparatus for sterilisation, wherein the sterilisation method comprises the use of a fluid at or near the supercritical pressure and temperature for that fluid. The MVU offers the various benefits of the invention as mentioned in this specification. The MVU provides additional confidence in the SCF sterilisation process and improved confidence in the sterilised substrates produced in the sterilisation process. As such, the confidence in the SAL is not solely reliant on the periodic calibration of the sterilisation process and apparatus to ensure that regulatory requirements are stringently met.

According to a third aspect of the invention, there is provided a sterilisation method comprising bringing a MVU and a material in need of sterilisation into contact with a sterilant fluid, the sterilant fluid comprising a fluid at or near the supercritical pressure and temperature for that fluid, and wherein the MVU is useful in evaluating the level of the sterilisation in the sterilisation method.

According to a fourth aspect of the invention, there is provided a sterilised material obtained by the sterilisation method of the third aspect of the invention, or an article derived thereof.

The MVU offers the benefit that the substrates treated under the conditions of the sterilisation process can be verified to a certain SAL, for example under conditions suitable to give a SAL of 10⁻⁶.

For the reasons mentioned earlier, quality control testing of a substrate to be sterilised which has been sterilised cannot do this. This is because substrates for human transplant can be substantially pre-sterilised prior to receiving a terminal sterilisation. The terminal sterilisation process used in SCF sterilisation is intended to give the assurance that sterilisation has occurred in the most stringent conditions possible, as is required by regulatory approval e.g. a SAL10⁻⁶.

In that regard, such substrates to be sterilised for human use are not deliberately contaminated (unlike the MVU) with an infectious species to ensure that for example 10⁶ infectious species are actually killed in the process. Hence, quality control testing of substrates which have been sterilised in a SCF sterilisation process can only show that the substrate is now presently sterile, and this does not give any information about the stringency of the conditions (e.g. the SAL) used to sterilise this substrate in the sterilisation process.

Therefore, by using the MVU in the sterilisation process it can be verified that the substrates have been sterilised to the same level of sterility as that experienced by a MVU sharing the sterilisation process.

The process calibration of the prior art cannot offer similar assurances about the sterilised substrate, as the calibration does not actually take place at the same time as the sterilisation of the substrates to be sterilised. Therefore, the MVU offers the benefit that the substrates to be sterilised (to a required SAL) have actually been verifiably exposed to the necessary sterilisation conditions required to bring about that level of sterilisation.

According to an embodiment of the fourth aspect of the invention, there is provided a sterilised material obtained by the sterilisation method according to the third aspect of the invention, or article derived thereof, wherein the sterilised material has been sterilised under conditions sufficient to achieve at least a 6-log or 12-log reduction, in a population of one or more CFUs.

According to a fifth aspect of the invention, there is provided a sterilised material obtainable by the third aspect of the invention, or article derived thereof, wherein the sterilised material has been sterilised under conditions sufficient to achieve at least a 6-log or 12-log reduction, in the population of one or more CFUs, and wherein this reduction is verifiable. The use of a MVU of the invention (e.g. first aspect of the invention) in the process of the invention (e.g. the third aspect of the invention) allows the sterilised substrates to be produced wherein it is verifiably demonstrated that these sterilised substrates where produced under conditions suitable to bring about a certain (challenging) degree of sterilisation, for example at least a 6-log reduction (i.e. SAL10⁻⁶), preferably at least a 12-log reduction (i.e. SAL10⁻¹²), in the population of one or more CFUs. A sterilised substrate which has been validated with a MVU in the SCF sterilisation process has a greater assurance of sterility than a substrate that was not sterilised with a MVU in the SCF sterilising process.

Preferably, the SAL to which the sterilised substrates can be verified to, will be equal to or greater than the SAL set by the regulatory body (e.g. a National, Regional or medical regulatory body) regulating these sterilised substrates. Preferably, the SAL will be twice (or greater than) the sterilisation level determined by the regulatory body regulating these sterilised substrates.

The use of a MVU allows the degree of sterilisation used in the formation (i.e. sterilisation) of the sterilised substrate to be verifiably determined i.e. the sterilisation conditions (used in the formation of the sterilised substrate) equates to that which was sufficient to bring about at least the measured reduction in the CFUs which were present in the MVU prior to sterilisation. That is, the MVU is intended to constitute a defined resistance to the sterilisation process.

According to a sixth aspect of the invention, there is provided a blister pack comprising two or more MVUs according to the first aspect of the invention, wherein the MVUs are detachable from the blister pack. A blister pack of MVUs allow for ease of use and a saving on manufacturing costs as compared to the making of individual MVUs.

The MVUs making up the blister pack should be readily detachable from the blister pack and so can be used as required. A blister pack can be manufactured so that it can be inserted as a whole in the sterilisation apparatus, thereby fixing the spatial relationship between each MVU as required by the user.

Preferably, the MVUs in the blister pack are detachable by means of a frangible section, such as a perforated strip.

According to a seventh aspect of the invention, there is provided a sealable gas-permeable container for use in a sterilisation method, wherein the sealable container comprises a MVU according to the first aspect of the invention.

In certain situations it may be desirable to have the MVU in close proximity to the substrate to be sterilised. This could take the form of a MVU having a separate sealable pouch which is adapted to house the packaged substrate to be sterilised. In use the packaged substrate to be sterilised is placed in the saleable container and the sealed container is sealed. In that way the substrate to be sterilised is held in close proximity to the MVU during the sterilisation process. The separate sealable pouch can be arranged to be easily separated from the MVU, e.g. by means of a frangible section, such as a perforated strip. Other arrangements will occur to the skilled person and are within the scope of the invention.

In this seventh aspect of the invention, it is possible for each substrate (to be sterilised) to be in effect equipped with a dedicated MVU.

According to an eighth aspect of the invention, there is provided a MVU kit for use in the evaluation of a sterilisation method, the kit comprising:

a sealable gas-permeable container, a sterilisation indicator medium, and a carrier medium, wherein the indicator medium comprises one or more structural features representative of the internal structure of the material to be sterilised in the sterilisation method, and wherein the carrier medium comprises a population of one or more CFUs, wherein the indicator medium and the carrier medium are combinable to form a sterilisation indicator.

The invention according to the eighth aspect of the invention provides for a kit assembly, such that the MVU can be assembled from its constituent parts. The kit can be conveniently prepared in advance or shortly before the MVU is to be used in a sterilisation process. The kit allows the nature of the MVU to be conveniently tailored to need. In that way, different options are readily available to the user, who may choose to vary the nature of the sterilisation indicator, the CFU species and/or the concentration of CFUs. The kit allows the MVU to be tailored to the substrate to be sterilised, so that the sterilisation process can be challenged appropriately by the MVU.

According to an ninth aspect of the invention, there is provided a method of manufacturing a MVU according to the first aspect of the invention comprising the step of housing the sterilisation indicator in the gas-permeable container, wherein the sterilisation indicator comprises an indicator medium and a population of one or more CFUs, and wherein the indicator, medium comprises one or more structural features representative of the internal structure of the material to be sterilised in the sterilisation method, and wherein the MVU is useful in evaluating the level of the sterilisation in the sterilisation method.

The invention provides for various ways in which the MVU may be manufactured. Preferably, the step of housing the sterilisation indicator within the gas-permeable container comprises heat-sealing of the gas-permeable container.

According to a tenth aspect of the invention, there is provided a method of manufacturing a sterilisation indicator as defined in the first aspect of the invention comprising the steps of: (i) optionally substantially sterilising the indicator medium; (ii) optionally pre-treating the indicator medium with a SCF; (iii) treating the indicator medium with a carrier medium, the carrier medium comprising a population of one or more CFUs, (iv) optionally incubating the treated indicator medium.

In order to establish the number of CFUs in the sterilisation indicator it can be preferable to start with a substantially sterilised indicator medium, and treat this medium with a given number of CFUs. Optionally this may involve an incubation step.

In addition it has been found that it is preferable to include a pre-treatment step, i.e. to pre-treat the indicator medium with a SCF, and wherein preferably the SCF is SCCD. This is substantially an additional cleaning/pre-conditioning step wherein protein and fatty materials and any other SCF soluble or dispersible materials are removed from the indicator material. This may in addition substantially pre-sterilise the indicator material prior to treatment with the inoculant containing the CFUs.

According to an embodiment of the tenth aspect of the invention, there is provided a method of manufacturing wherein the indicator medium is pre-treated with a SCF, or wherein the indicator medium is pre-treated with a SCF and wherein the SCF is SCCD.

In addition, the pre-treatment step removes 25 to 99% of the mass of the material pre-treated, preferably 70 to 98% of the mass of material pre-treated, more preferably 85 to 95% of the mass of the material pre-treated in the pre-treatment process.

The number of CFUs in the colony is often considered to be the minimum number of units in that colony and so it is possible in some circumstances to omit a pre-sterilisation step prior to inoculation with the CFUs.

Methods of incubating CFUs are well known in the art and are not discussed here. Similarly, methods of determining the level of CFUs in a sample are also well known and are not discussed here.

According to an eleventh aspect of the invention, there is provided the use of a sterilised material, or article derived thereof, according to the fourth or fifth aspects of the invention in a method of medical treatment.

The use of a sterilised product according to the invention is also considered. Examples of suitable materials or articles include biological tissue for transplants, allografts, xenografts, implants, articles useful in bodily reconstruction, internal scaffolding, sutures, diagnostic probes, instrumentation, drug release bodies, or articles which act as conduits to the body and other similarly related sterilised articles. Particularly considered are sterilised articles which must meet a high regulatory standard prior to being used on or inserted into the human body. More particularly considered are where the regulatory standard mentioned above requires at least a 6-log (SAL10⁻⁶), or 12-log (SAL10⁻¹²), reduction in the population of one or more CFUs.

According to a twelfth aspect of the invention, there is provided a method of medical treatment comprising the use of a sterilised material, or article derived thereof, according to the fourth or fifth aspects of the invention. The use of a sterilised product according to the invention is also considered in methods of medical treatment, including but not limited to the use in transplantation, implantation, reconstruction, internal scaffolding, diagnostic instrumentation, bodily conduits and in surgery.

According to a thirteen aspect of the invention, there is provided use of a sterilised material, or article derived thereof according to the fourth or fifth aspects of the invention in the manufacture of a product for use in implantation or transplantation.

According to an embodiment of the first aspect of the invention, preferably, the gas-permeable container is substantially gas-permeable over its entire surface.

It is required that the SCF can penetrate into the container housing the sterilisation indicator. It is preferable that the SCF can enter the container housing the sterilisation indicator substantially across the entire surface area of the MVU, to allow efficient entry of the sterilant.

Optionally, the container housing the sterilisation indicator could be gas-permeable over only a portion of the surface area of the housing (e.g. one or more gas-permeable windows). This might be done for example to present a greater challenge to the sterilisation process.

Typically, the sterilisation indicator and the substrate to be sterilised will be housed in substantially the same kind of container, e.g. inner and outer containers each being made of a gas-permeable non-woven polymeric material.

According to an embodiment of the first aspect of the invention, preferably the container comprises Tyvek, more preferably 1073B Tyvek. Preferably, the container comprises a material substantially the same as 1073B Tyvek as manufacture and sold in 2012. Tyvek is a commercially available polymeric material which is durable with good gas-permeability and which can be purchased in pouches ready for convenient sealing.

According to an embodiment of the first aspect of the invention, preferably, the container comprises a non-woven bag or pouch comprising 1073B Tyvek.

According to an embodiment of the first aspect of the invention, preferably, the MVU is housed within a second gas-permeable container.

The second (outer) container is usually made from the same material as the first (inner) container, with the first (inner) container housing the sterilisation indicator. The second container may be housed in one or more further gas-permeable containers.

The second (or further containers) can act as a fail-safe in case the first gas-permeable container should unexpectedly fail. Such a failure could result in the contamination of the sterilisation apparatus and the general working environment. The second (and/or further containers) can be arranged to resist the entry of the SCF, so as to present a greater challenge to the sterilisation process as required. The further containers (if present), like the second outer container, can be gas-permeable, porous (i.e. not necessary just gas-permeable) or could be an open container, as determined by the needs of the user.

The outer gas-permeable container can have the dimensions of about 7×10 cm. The inner gas-permeable container can have dimensions of about 5×8 cm. However, the innermost container can be appropriately sized to accommodate the sterilisation indicator, and the outermost container can be appropriately sized to accommodate the inner container. Typically the gas-permeable containers can have a pouch-like structure and so can expand to accommodate the materials put within.

The one or more gas-permeable containers are preferably heat sealed, however any suitable means of sealing the gas-permeable containers is considered. Optionally, the atmosphere in the Tyvek pouch is filled with an inert gas.

According to an embodiment of the first aspect of the invention, preferably, the indicator medium of the sterilisation indicator comprises a porous structure.

A porous indicator medium presents a greater challenge to the SCF sterilisation process than a non-porous medium (i.e. substantially non-gas permeable), as the SCF is required to penetrate into the internal structure of the indicator medium. Also a porous structure has a greater surface area to sterilise than a non-porous structure. Furthermore, a porous structure better mimics the internal structure of biological tissue such as bone. A porous indicator medium is more representative of biological substrates (to be sterilised), and so can better challenge the sterilisation process used.

The MVU (to challenge the sterilisation process adequately) should comprise one or more structural features which mimic the physical nature of the substrates to be sterilised. Preferably, the MVU should comprise a surface area and/or internal physical structure which mimics the nature of the substrate to be sterilised.

This is to ensure that MVU best mimics the substrates to be sterilised and most particularly simulates those places in which an infectious species might inconveniently and inaccessibly reside.

For example, biological tissue like bone has a porous (i.e. lattice-like internal structure), with a correspondingly large internal surface area. This honey-comb like surface can have many inaccessible regions. These inaccessible regions can harbour infectious agents and in effect could shield infectious agents from the sterilant used. Accordingly, porous structures with large internal surface areas can be difficult to fully treat with a sterilant.

A simple (non-porous) plate harbouring a colony of microorganisms on its outer surface cannot properly mimic the challenging conditions faced when sterilising biological tissue like bone. A (non-porous) plate of this kind does not sufficiently challenge the sterilisation process. It is difficult to therefore have confidence that the sterilisation conditions used to sterilise a simple glass plate would be sufficiently rigorous to equally sterilise a porous material like human bone.

Preferably the MVU will have a surface area which is substantially the same or greater than the substrate to be sterilised.

Preferably, the physical nature of the MVU should be more challenging to sterilise than the physical nature of the substrate to be sterilised.

According to an embodiment of the first aspect of the invention, preferably, the indicator medium comprises one or more materials selected from: tissue suitable for transplant, research or therapeutical grade bone, demineralised bone, demineralised bone matrix, a paste comprising demineralised bone or demineralised bone matrix, whole cortical bone pieces, tendon, Achilles Tendon, cartilage, ligament, skin, connective or musculoskeletal tissue or biological tissue suitable for implantation; natural or synthetic polymer, biomedical polymer, medical-grade polymer, biodegradable polymer, synthetic human tissue analogue; biologically active molecules, pharmaceutically active compounds, pharmaceutical carriers, or pharmaceutical delivery vehicles; metals, alloys, medical equipment, instruments, prosthetics, implants or representative analogues thereof.

The indicator medium of the sterilisation indicator is preferably selected to correspond to the substrates to be sterilised. Ideally the indicator medium to be sterilised should have physical properties which are the same or correspond closely to the substrate to be sterilised. Conceivably, the indicator medium can be made of the same material as the substrate to be sterilised.

However, therapeutical grade bone (i.e. suitable for human transplantation) is a scarce resource and so would not be typically used for making indicator mediums. Accordingly, it is preferred to use a material that mimics the physical properties of the therapeutical grade material to be sterilised, so as to preserve this scarce resource for actual transplantation. However, in certain circumstance transplant grade tissue can be used in the indicator medium of the MVU.

According to an embodiment of the first aspect of the invention, preferably, the indicator medium comprises Cortical Cancellous Crunch.

The Cortical Cancellous Crunch can comprise about 60% to 90% Cancellous bone, preferably 70 to 85 Cancellous bone and more preferably 75 to 80% Cancellous bone. In addition the Cortical. Cancellous Crunch can comprise about 10 to 40% Cortical bone, preferably 15 to 30% Cortical bone and more preferably 20 to 25% Cortical bone. More preferably still, Cortical Cancellous Crunch comprises about 75 to 80% Cancellous bone and 20% to 25% Cortical bone.

Cortical Cancellous Crunch is a porous bone-derived product. Cortical Cancellous Crunch has an internal structure which corresponds well to the internal structural features of many other bone-types which are routinely used in transplantation. As Cortical Cancellous Crunch has a porous physical structure it presents a challenge to normal sterilisation processes.

According to an embodiment of the first aspect of the invention, preferably, the indicator medium comprises human-derived tissue.

The use of human-derived tissue as the sterilisation indicator medium offers the closest facsimile to the human substrates which are to be sterilised.

The sterilisation indicator medium may include tissue suitable for transplant, research or therapeutical grade bone, demineralised bone, demineralised bone matrix, a paste comprising demineralised bone or demineralised bone matrix, whole cortical bone pieces, tendon, Achilles tendon, cartilage, ligament, skin, connective or musculoskeletal tissue or biological tissue suitable for implantation, or other biologically derived materials suitable for implantation; natural or synthetic polymer, biomedical polymer, medical-grade polymer, biodegradable polymer, synthetic human tissue analogue; biologically active molecules, pharmaceutically active compounds, pharmaceutical carriers, or pharmaceutical delivery vehicles; metals, alloys, medical equipment, instruments, prosthetics, implants or representative analogues thereof. The sterilisation indicator is preferably the same kind of human-derived tissue as the human tissue to be sterilised in the sterilisation process. Preferably, the indicator medium comprises research grade bone. More preferably, the indicator medium should be tissue closely matching (or be of the same kind of tissue) as the material to be sterilised in the SCF sterilisation process.

However, it is also conceived that the sterilisation indicator medium could be derived from an animal species (i.e. a xenogenic-sourced material). For example bovine-sourced or porcine-sourced material (e.g. large boned animals) could be used as the source of the sterilisation indicator medium. It is also conceived that these animals could be genetically modified to contain human genetic material.

The sterilisation indicator is preferably formed of 15 gram portions of human-derived Cortical Cancellous Crunch in a granular form. However, 0.25, 0.5, 1, 2, 2.5, 3, 4, 5, 6, 7, 7.5, 8, 9, 10, 11, 12, 13, 14, 15, 16, 17, 18, 19 and 20, 25, 30, 50, 75 and 100 gram portions are also considered. Preferably the indicator medium should be approximately or substantially the same mass as the material to be sterilised in the SCF sterilisation process.

Preferably, the Cortical Cancellous Crunch is research grade bone.

Also considered are non-biologically derived indicator mediums which comprise one or more structural features representative of the internal structure of the material to be sterilised in the SCF sterilisation method.

According to an embodiment of the first aspect of the invention, preferably, the synthetic tissue analogue comprises silica, glass or a ceramic material.

In certain situations it would be preferable for the sterilisation indicator to comprise a synthetic tissue analogue material which mimics one or more of the physical or structural features of the substrate to be sterilised. For example, the sterilisation indicator might comprise a porous glass or ceramic material with a large internal surface area. This porous structure substantially mimicking the internal surface structure of human bone.

These non-biologically derived indicator mediums might include, natural or synthetic polymer, biomedical polymer, medical-grade polymer, biodegradable polymer, metals or alloys.

These non-biologically derived indicator mediums might also include biologically active molecules, pharmaceutical carriers or pharmaceutical delivery vehicles.

A durable synthetic material offers the possibility that after use, it can be recycled. That is, it could be cleaned, sterilised and re-inoculated with a colony of CFUs ready for reuse in another MVU. Accordingly, any extra cost associated with the manufacture of such a synthetically-derived sterilisation indicator could be offset by multiple use.

According to an embodiment of the first aspect of the invention, preferably, the indicator medium is granular, powder or fibrous in nature and/or formed into a solid mass

Preferably, the sterilisation indicator is substantially a granular mixture. However, the indicator medium can take any reasonable form including a moulded or compacted product, or could be in a fibrous form, or in a loosely woven structure.

Such granules or fibres might also be contained in a convenient retaining structure such as an open glass vial or simply tied together when fibrous.

According to an embodiment of the first aspect of the invention, preferably, the indicator medium comprises granules of between about 1 and 9 mm, 2 and 7 mm, or 3 and 5 mm, and where the indicator medium comprises a powder, the powder has a mean particle diameter of about 10 to 900 μm, 100 to 700 μm, or 150 to 350 μm.

The sterilisation indicator of the MVU will comprise a population of one or more CFUs.

According to an embodiment of the first aspect of the invention, preferably, the population of the one or more CFUs is at least 10³ CFUs, 10⁶ CFUs or 10¹² CFUs. Preferably the population of the one or more CFUs comprises 2×10⁶ CFUs.

The MVU can be inoculated with a known amount of a particular microorganism. In that way the sterilisation process can be challenged under the most rigorous conditions possible and the level of sterilisation can be directly measured from the sterilised colony.

Preferably, the indicator medium will comprise over 10⁶ CFUs. Sterilising 10⁶ CFUs in an indictor medium gives a SAL of at least 10⁻⁶ for a substrate that it substantially sterile prior to sterilisation. SAL10⁻⁶ is a sterilisation level set by many National regulatory bodies for substrates which are to be used in human surgery.

Preferably the indicator medium with comprise over 10¹² CFUs. Sterilising 10¹² CFUs in an indictor medium gives a SAL of at least 10⁻¹² for a substrate that it substantially sterile prior to sterilisation.

According to an embodiment of the first aspect of the invention, preferably, the one or more CFUs are selected from bacteria, vegetative microbial cells, moulds, single-celled organisms, protozoa, yeasts, viruses, or other infective agents, spores, or progenitor species thereof.

The CFUs can be selected from any suitable microorganism (or progenitor species thereof inclusive of viral species), but is preferably selected from those which have been known to be difficult to sterilise in the past. Bacterial spores for example are preferred as they represent a significant challenge to most sterilisation processes.

According to an embodiment of the first aspect of the invention, preferably, the one or more CFUs are selected from B. Atrophaeus (formerly B. subtilis var Niger), B. stearothermophilus, B. subtilis, B. pumilus, B. cereus, Listeria innocua, Staphylococcus aureus, Salmonella salford, Psuedomonas aeruginosa, Escherichia coli, Preoteus vulgaris, Legionella dunnifii; spores, or progenitor species thereof, or wherein the one or more CFUs is a 2×10⁶ B. Atrophaeus spore suspension.

The preferred CFU species mentioned above are species which have been shown to have increased resistance to sterilisation. Accordingly, these CFU species represent a significant challenge to the sterilisation process.

The infectious species used in the sterilisation indicator can be selected appropriately to challenge the sterilisation process, even selecting an infectious species that goes beyond that which might be typically expected to be found in the substrates to be sterilised.

A species that challenges the sterilisation process gives good confidence that other more likely (and less robust) contaminating species will be successfully sterilised as well in a sterilisation process.

Preferably, the CFUs are substantially evenly distributed throughout the structure of the sterilisation indicator medium.

Preferably, the CFUs are a colony of at least 10⁶ B. Atrophaeus (formerly B. subtilis var Niger), B. stearothermophilus, or 10⁶ B. subtilis. These colonies represent a significant challenge to a SCF sterilisation process. More preferably, the CFU are B. Atrophaeus (formerly B. subtilis var Niger), spores thereof, or a mixture of thereof. Most preferably the CFUs are a colony at least 2×10⁶ B. Atrophaeus (formerly B. subtilis var Niger) spores.

According to an embodiment of the invention, there is provided a sterilisation method according to the third aspect of the invention wherein the MVU is a MVU according to the first aspect of the invention.

In use one or more MVUs are placed in a sterilisation apparatus together with the substrate to be sterilised. The substrate to be sterilised and the MVU are treated under the conditions required to bring about sterilisation, preferably under conditions suitable to give a desired SAL, e.g. a SAL10⁻⁶.

The one or more MVUs are separated from the substrates to be sterilised. The one or more MVUs are tested to ascertain the level of sterilisation achieved in the sterilisation process.

The evaluation of the level of sterilisation would typically involve removing the sterilisation indicator from the MVU and subjecting this to testing in accordance with standard evaluation technique (e.g. the British Pharmacoepia method), optionally including incubating of the sterilisation indicator to promote the growth of any remaining live CFUs prior to testing.

If no CFUs persist (and so cannot have been incubated) then the level of sterilisation can be reliably determined. For example, where the sterilisation indicator was inoculated with 10⁶ CFUs prior to sterilisation, then where all CFUs are destroyed in the sterilisation process, then the level of sterilisation can be reliably verified as sufficient to bring about this level of reduction in CFUs.

For example, to achieve a sterilisation level equivalent to the destruction of at least 10¹² CFUs then a sterilisation indicator harbouring a colony of 10¹² bacteria (or other infectious species) could be used. Clearly the colony of CFUs can be tailored to suit need, e.g. 10¹, 10², 10³, 10⁴, 10⁵, 10⁶, 10⁷, 10⁸, 10⁹, 10¹⁰, 10¹¹ or greater than 10¹¹ CFUs.

In use, it is preferred that at least one MVU of the invention is retained as a control specimen and as such not treated under the conditions of the sterilisation method. This control specimen can then be compared to the MVU(s) which were subjected to the sterilisation process.

Where incomplete sterilisation of the MVU has occurred in the sterilisation method, the control specimen may allow the degree of sterilisation to be calculated. However complete sterilisation of the CFUs is normally desired as this represents the simplest way to calculate the minimum SAL level.

The control specimen (i.e. positive control) ensures that the MVU used in the sterilisation process was not in some way defective prior to sterilisation in the sterilisation process. To ensure reliability, the control MVU specimen and the MVU used in the sterilisation process should preferably be from the same batch.

According to an embodiment of the third aspect of the invention, preferably, the sterilisation method is a terminal sterilisation method.

A terminal sterilisation process is a sterilisation process which occurs as the final sterilisation step in the process of producing a substrate to be sterilised. After terminal sterilisation, typically no further sterilisation of the substance to be sterilised is required before actual use in treatment or surgery. It is preferable that the terminal sterilisation process is the most stringent sterilisation process. Prior to use the sterilised article is removed from the terminal sterilisation packaging, taking normal surgical precautions in handling such sterile items.

According to an embodiment of the third aspect of the invention, preferably, the fluid in the sterilisation method is carbon dioxide.

The MVU of the invention is particularly suited to the sterilisation method of the invention where the SCF is SCCD. Carbon dioxide has been shown to be particularly effective in the sterilisation of sterilisation-resistant microorganisms in a gentle but effective way. However, the MVU would be compatible with other sterilisation methods using other SCFs. Examples of such SCFs inclusive of critical temperature and pressures are provided in Table 1.

TABLE 1
Critical properties of various SCF (Reid et al., 1987)

| | Molecular | Critical | Critical | Critical |
|---|---|---|---|---|
| | weight | temperature | pressure | density |
| SCF | g/mol | K | MPa (atm) | g/cm³ |
| Carbon dioxide | 44.01 | 304.1 | 7.38 (72.8) | 0.469 |
| (CO₂) | | | | |
| Water (H₂O) | 18.015 | 647.096 | 22.064 | 0.322 |
| | | | (217.755) | |
| Methane (CH₄) | 16.04 | 190.4 | 4.60 (45.4) | 0.162 |
| Ethane (C₂H₆) | 30.07 | 305.3 | 4.87 (48.1) | 0.203 |
| Propane (C₃H₈) | 44.09 | 369.8 | 4.25 (41.9) | 0.217 |
| Ethylene (C₂H₄) | 28.05 | 282.4 | 5.04 (49.7) | 0.215 |
| Propylene (C₃H₆) | 42.08 | 364.9 | 4.60 (45.4) | 0.232 |
| Methanol (CH₃OH) | 32.04 | 512.6 | 8.09 (79.8) | 0.272 |
| Ethanol (C₂H₅OH) | 46.07 | 513.9 | 6.14 (60.6) | 0.276 |
| Acetone (C₃H₆O) | 58.08 | 508.1 | 4.70 (46.4) | 0.278 |

Preferably the SCF should be substantially pure prior to use.

According to an embodiment of the third aspect of the invention, preferably, the sterilant fluid comprises one or more additives selected from: a peroxide, carboxylic acid, acid anhydride, ester or alcohol, or is a commercial additive package. Preferably the additive package is NovaKill. Preferably the one or more additives are selected from trifluoroacetic acid, peracetic acid, acetic acid, hydrogen peroxide, Trifluoroacetic anhydride, acetic anhydride, citric acid, succinic acid, formic acid, malonic acid, phosphoric acid, ethyl acetate or ethanol. Optionally, the additive is a hydrolysable precursor to one or more of the above additive species. More preferably the additive package is NovaKill (trade name of a SCCD additive package sold by NovaSterilis), or a composition having the same, or substantially the same, composition as the NovaKill additive package as manufacture and sold in 2012.

Additives have been shown to improve the effectiveness of a SCF sterilisation process. For example the sterilisation process using SCCD has been shown to be improved by the incorporation of organic acids, compounds that hydrolyse to organic acids or peroxides.

According to an embodiment of the third aspect of the invention, preferably, the additive is present in an amount of between about 0.001% to about 2.0% based on the total volume of the sterilant fluid. More preferably the additive is present in an amount of between about 0.05% to about 1.5% based on the total volume of the sterilant fluid, and most preferably between about 0.5% to about 1.0% based on the total volume of the sterilant fluid.

According to an embodiment of the invention, there is provided a sterilisation method according to the third aspect of the invention, wherein the material in need of sterilisation is from a human donor.

According to an embodiment of the invention, there is provided a sterilisation method according to the third aspect of the invention, wherein the material in need of sterilisation is from one or more donors.

According to an embodiment of the invention, there is provided a sterilisation method according to the third aspect of the invention, wherein the material in need of sterilisation is from one or more live donors or from one or more cadaveric donors or is a mixture of live and cadaveric donors.

According to an embodiment of the invention, there is provided a sterilisation method according to the third aspect of the invention, wherein the sterilisation chamber is 20 to 200 litres, 60 to 150 litres, or 80 to 100 litres.

There is a prejudice in the art that teaches away from using donor tissue obtained from live donors (e.g. femoral head tissue, e.g. from hip replacement operations or metaphyses of long bones resulting from amputation) as the material to be sterilised in a SCF sterilisation process.

In addition there is prejudice in the art that teaches away from using materials obtained from more than one donor as the material in need of sterilisation in the same sterilisation process.

In addition there is prejudice in the art that teaches away from using the material in need of sterilisation which has been obtained from live donors and cadaveric donors in the same sterilisation process/batch.

In addition there is prejudice in the art that teaches away from using the material in need of sterilisation which has been obtained by mixing tissue of live donors and cadaveric donors in the same sterilisation process/batch.

This prejudice stems from the extremely cautious and conservative nature of the medical practitioners and regulatory bodies responsible for approving such sterilised materials for use in human surgery. Such practitioners and regulatory bodies are very reluctant to expose patients to medical risk without reliable and verifiable indications that such new practices are safe.

The MVU provides a solution to that problem. The MVU provides the means to verify that the sterilisation process experience by the material in need of sterilisation (regardless of source) have met the minimum SAL requirement required by those regulatory bodies (e.g., a SAL of 10⁻⁶). That is, the MVU allows such new sterilisation techniques to therefore be verified to the satisfaction of the medical/regulatory bodies.

The MVU therefore provides at least the advantage of the access to new sources of donor material i.e. from live donors, at least within a cost effective manner.

In addition the MVU allows material from more than one donor to be treated in the same (i.e. single) sterilisation process/batch. At the present time, because material from only a single donor can be sterilised at one time, this puts a limit on the quantity of the material that can be sterilised in one batch (i.e. about 20 to 30 grams per femoral head from a live donor, or about 300 grams from an average cadaveric donor using a 20 litre sterilisation chamber). However, when material obtained from more than one donor can be sterilised in a single batch (i.e. pooled), this would allow batches of greater sizes to be sterilised (e.g. 300 grams to 600 grams using a 20 litre sterilisation chamber). Indeed, this pooling would allow access to the use of larger sterilisation chambers, which could then be used to sterilise the material from several cadaveric donors in a single batch. Therefore, this pooling would result in a saving in costs and time due to economies of scale.

Furthermore, as there is currently an expectation in the art that only the material from a single donor can be treated in a SCF sterilisation process at one time, this has led to the expectation in the art that sterilisation chambers of only a limited volume are required (typically up to 20 litres). However, the MVU provides access to the processing of greater quantities of materials, which leads to the demand for larger sterilisation apparatus having sterilisation chambers of greater volume. Accordingly, the MVU provides an incentive for the development and innovation of larger sterilisation apparatus with sterilisation chambers of greater volumes (e.g. 10, 20, 40, 50, 60, 100, 150 or 200 L sterilisation chambers), where otherwise such incentive would be lacking in that field of industry.

Accordingly, the MVU allows for more efficient sterilisation processes, reduction in costs and time associate with economies of scale, greater flexibility in the materials (and sources of those materials) that can be processed together in a single process and access to new sources of human tissue, resulting in less waste of a valuable and scarce resource (i.e. transplant grade human donor material).

In a larger SCF sterilisation chamber, a greater number of MVUs could be employed than in a smaller sized chamber.

According to an embodiment of the fourth or fifth aspect of the invention, preferably, the sterilised material or article derived thereof, is selected from therapeutic grade tissue suitable for transplant, bone, demineralised bone, demineralised bone matrix, a paste comprising demineralised bone or demineralised bone matrix, whole cortical bone pieces, tendon, Achilles tendon, cartilage, ligament, skin, connective or musculoskeletal tissue or biological tissue suitable for implantation; natural or synthetic polymer, biomedical polymer, biodegradable polymer, synthetic human tissue analogue; biologically active molecules, pharmaceutically active compounds, pharmaceutical carriers, or pharmaceutical delivery vehicles; metals, alloys, medical equipment, instruments, prosthetics or implants.

According to an embodiment of the fourth or fifth aspect of the invention, preferably the sterilised material or article derived thereof comprises Cortical Cancellous Crunch.

The Cortical Cancellous Crunch can comprise about 60% to 90% Cancellous bone, preferably 70 to 85 Cancellous bone and more preferably 75 to 80% Cancellous bone. In addition the Cortical Cancellous Crunch can comprise about 10 to 40% Cortical bone, preferably 15 to 30% Cortical bone and more preferably 20 to 25% Cortical bone. More preferably still, Cortical Cancellous Crunch comprises about 75 to 80% Cancellous bone to and 20% to 25% Cortical bone.

The above materials or articles are examples, and other suitable materials or articles are considered.

According to an embodiment of the fourth or fifth aspect of the invention, preferably, the article derived from the sterilised material is selected from an allograft, implant, stent, catheter, endoscope, prosthesis, joint replacement, medical scaffolding, suture, medical instrument, surgical instrument, drug delivery device or pharmaceutically active implant or microparticles.

The above materials or articles are examples, and other suitable materials or articles are considered.

According to a fourteenth aspect of the invention, there is provided a pre-treatment step of pre-treating a material to be sterilised with a SCF, wherein the material to be sterilised include those defined in the fourth and fifth embodiment of the invention, and preferably wherein the SCF is SCCD.

According to a fifteenth aspect of the invention, there is provided a pre-treatment step of pre-treating an indicator medium, and/or pre-treating a material to be sterilised, with a SCF, wherein the indicator medium material and/or the material to be sterilised is/are those defined in any one of the aspects of the invention, and preferably wherein the SCF is SCCD.

A MVU according to the first aspect of the invention, wherein the indicator medium has been pre-treated with a SCF, or wherein the indicator medium has been pre-treated with a SCF and wherein that SCF was SCCD. More preferably, wherein pre-treatment has removed 25 to 99% of the mass of the material pre-treated, or has removed 70 to 98% of the mass of the material pre-treated, or has removed 85 to 95% of the mass of material pre-treated.

A method of manufacturing a sterilisation indicator as defined in the tenth aspect of the invention, comprising the steps of: (i) optionally substantially sterilising the indicator medium; (ii) optionally pre-treating the indicator medium with a SCF; (iii) treating the indicator medium with a carrier medium, the carrier medium comprising a population of one or more CFUs, (iv) optionally incubating the treated indicator medium. Preferably, wherein the indicator medium is pre-treated with a SCF, and wherein preferably the SCF is SCCD. More preferably, wherein pre-treatment removes 25 to 99% of the mass of the material pre-treated, or removes 70 to 98% of the mass of material pre-treated, or removes 85 to 95% of the mass of the material pre-treated.

A sterilisation method according to the third aspect of the invention, wherein the material in need of sterilisation has been pre-treated with a SCF, or wherein the material in need of sterilisation has been pre-treated with a SCF and wherein that SCF was SCCD. More preferably, wherein the pre-treatment has removed 25 to 99% of the mass of the material pre-treated, or has removed 70 to 98% of the mass of the material pre-treated, or has removed 85 to 95% of the mass of the material pre-treated.

Pre-treatment (i.e. a treatment process occurring prior to the SCF sterilisation process) of a material to be sterilised (and/or of an indicator medium) has the advantage that proteins, fatty materials (e.g. fats, lipids and greases) and any other SCF-extracted materials (inclusive of SCF-soluble materials) are removed from the material being pre-treated. These SCF-extracted materials are typically trapped on medical gauze during the pre-treatment step. However, other means of trapping these SCF-extracted materials can be undertaken, such as the use of one or more filters which can be placed in the SCF pre-treatment chamber, or could be located remotely from that chamber (for example in line with the impellor). This SCF pre-treatment may in addition substantially pre-sterilise the material being treated. However, normally, the conditions used in the SCF pre-treatment step are not normally sufficient to bring about a terminal sterilisation which would meet the required regulatory standards. Pre-treatment may be considered a de-lipidisation or cleaning step.

In the case of the materials to be sterilised (including those defined in the fourth or fifth aspect of the invention), the removal of the SCF-extracted materials from the material to be sterilised provides various advantages to the resultant pre-treated product. These advantages include increased bone growth (i.e. bone in-growth) between the material transplanted and the bone of the patient. This leads to faster recovery of the patient. In addition, the increased bone in-growth provides a more secure bonding of the transplanted material to the bone of the patient. This leads to a stronger resultant incorporation of the transplanted material. This leads to resultant bone which is less likely to suffer a structural failure as compared to bone incorporating non pre-treated materials. In addition, the removal of donor proteins (and any other biological matter) from the material to be transplanted reduces the risk that there will be some form of immune system rejection of the transplanted material. The pre-treatment step also means that (due to the removal of extraneous materials from the material to be sterilised), that the SCF can better penetrate into the materials to be sterilised.

When the material to be sterilised is pre-treated with a SCF, it is preferable to also pre-treat the indicator medium of the MVU in substantially the same way. This allows the indicator medium to best replicate the material to be sterilised.

According to a sixteenth aspect of the invention, there is provided a pre-treatment method, wherein a material in need of sterilisation is pre-treated with a SCF, or wherein the material in need of sterilisation is pre-treated with a SCF and wherein that SCF is SCCD.

The material obtained by the pre-treatment method of the sixteenth aspect of the invention is useful in the MVU sterilisation process of the invention, but this pre-treated material also has application in other SCF and non-SCF sterilisation processes. These SCF and non-SCF processes will be known to the skilled person, and include chemical sterilisation (e.g. using ethylene oxide or steam) and/or sterilisation by irradiation and/or radiation (e.g. UV and gamma rays).

The pre-treated material according to the sixteenth aspect of the invention, wherein preferably, the pre-treatment method has removed 25 to 99% of the mass of the material pre-treated, or has removed 70 to 98% of the mass of the material pre-treated, or has removed 85 to 95% of the mass of the material pre-treated.

Also considered is a sterilisation method comprising bringing the pre-treated material obtained/obtainable from the sixteenth aspect of the invention, in need of sterilisation into contact with a sterilant fluid, wherein optionally the sterilant fluid comprises a fluid at or near the supercritical pressure and temperature for that fluid. Preferably that SCF is SCCD. Preferably, wherein the sterilisation process includes one or more additives as defined in an embodiment of the third aspect of the invention. For the reasons given above, the pre-treated material (i.e. which has had lipids and protein materials removed by the SCF) has improved properties when used in transplantation, as compared to the equivalent material in which no pre-treatment with a SCF has been used.

### BRIEF DESCRIPTION OF THE DRAWINGS

Two preferred embodiments of the MVU of the first aspect of the invention will now be described, by way of example only, with reference to the accompanying drawings wherein:

FIG. 1 is a plan view of an embodiment of the MVU of the first aspect of the invention.

FIG. 2 is a plan view of a further embodiment of the MVU of the first aspect of the invention.

FIG. 3 is a schematic diagram of a SCF sterilisation apparatus useful in SCF sterilisation process according to the third aspect of the invention.

### DETAILED DESCRIPTION OF THE PREFERRED EMBODIMENTS

In the drawings, like parts are given the same reference numerals.

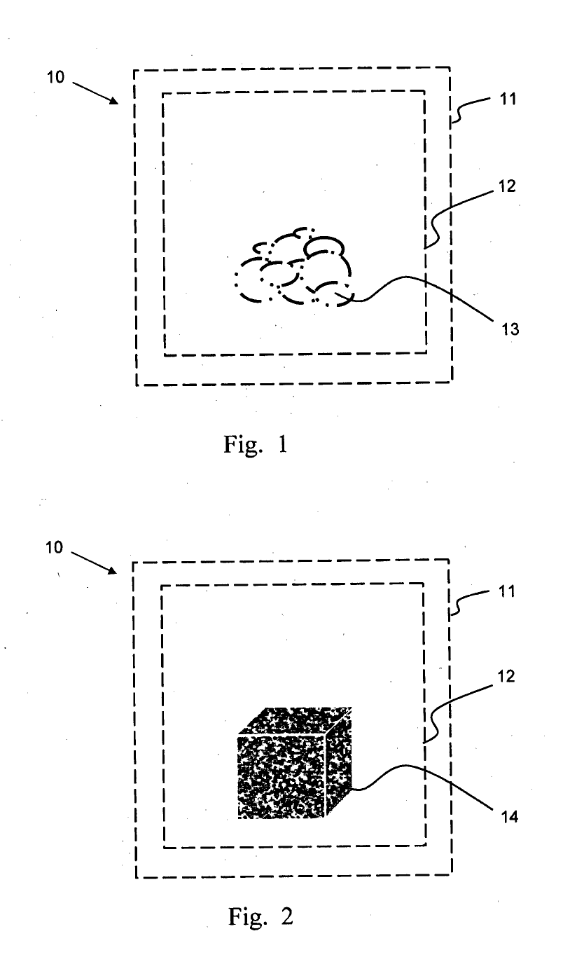

FIG. 1 shows a schematic diagram of a preferred embodiment of a MVU 10 of the first aspect of the invention.

In FIG. 1 the MVU 10 includes an outer gas-permeable container 11, an inner gas-permeable container 12 and sterilisation indicator 13.

The gas-permeable containers 11 and 12 shown in FIG. 1 comprise a material that is permeable to the SCF to be used.

However, it should be understood that the outer container 11 may not be required or could be replaced with a porous material (i.e. a material which is not necessary only gas-permeable), or could be replaced with an open container, as determined by the requirements of the user.

The sterilisation indicator 13 in FIG. 1 is human-derived research grade Cortical Cancellous Crunch in a granular form.

FIG. 2 shows a schematic diagram of a further embodiment of the MVU of the first aspect of the invention which corresponds to the embodiment shown in FIG. 2 except that the indicator medium of the sterilisation indicator 14 is a synthetic tissue analogue, shown here as a porous ceramic cube.

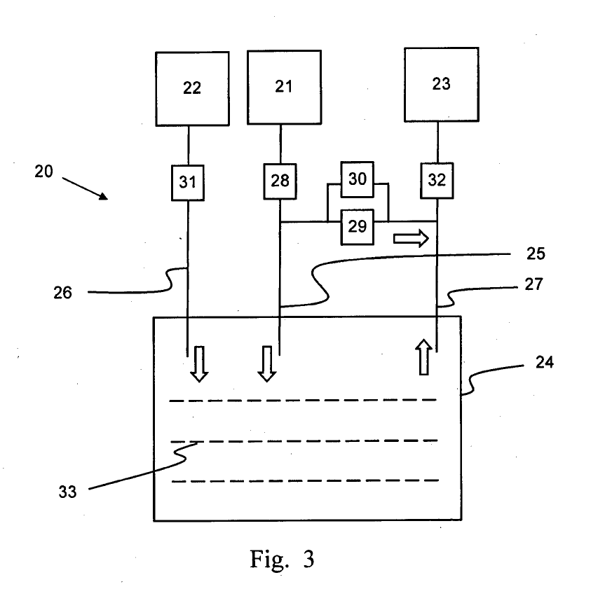

FIG. 3 shows a schematic diagram of a SCF sterilisation system 20 for use in a method according to the invention comprising a MVU.

In this regard, it can be seen that the sterilisation system 20 includes a fluid source unit 21, an (optional) additive source unit 22, an outlet unit 23 and a sterilisation vessel 24.

The fluid source unit 21 provides a gas or fluid to the sterilising vessel 24 via inlet line 25.

The fluid source unit 21 and inlet line 25 allow the gas (which is to form the SCF, e.g. carbon dioxide) to be introduced into the sterilisation vessel 24 in a controlled manner.

The fluid source unit 21 can be made up of a compressed gas cylinder (for example pressurised cylinder containing carbon dioxide, or another suitable gas which is capable of forming a SCF) and a standard air compressor. A compressed gas cylinder can optionally be used in conjunction with a gas booster (for example a Haskel Booster AGT-7/30). Alternatively, the air compressor and booster can be replaced with a single gas compressor. It is also possible that the SCF could be condensed in a separate container and fed to the sterilisation vessel via an input line. Preparing a SCF in a separate container could reduce the induction time used in the sterilisation process.

The inlet line 25 may optionally be equipped with one or more monitoring, purifying or control means, these being shown schematically as 28. These include, but are not limited to pressure gauges, flow meters, valves and filters.

Inlet line 25 may also optionally be equipped with an isolatable exit line (also optionally equipped with one or more monitoring, purifying or control means) as schematically shown as 29.

Inlet line 25 may also optionally be equipped with a pressure relief module 30. The pressure relief module 30 is arranged to prevent over pressurisation of the system. This pressure relief module 30 may for example contain a pressure relief valve which is trigger when the pressure exceeds a predetermined value. The pressure relief valve may be automatically or manually triggered, or could be triggered in response to an active monitoring system (not shown).

The additive source unit 22 can provide one or more additives to the sterilisation vessel 24 via an inlet line 26. The inlet line 26 may optionally be equipped with one or more monitoring, purifying or control means, these being shown schematically as 31. These include, but are not limited to pressure gauges, flow meters, valves and filters.

The additive source unit 22 and inlet line 26 allows one or more additives (e.g. peroxide or carboxylic acid, or other agent that beneficially improves the sterilisation process) to be introduced into the sterilisation vessel, preferably in a measurable and controlled manner. As require, one or more additive source units could be provided, or a single module could be used to introduce more than one additive.

An outlet unit 23, via outlet line 27, allows gas and/or fluid products to be removed from the sterilisation vessel 24. The outlet line 27 may optionally be equipped with one or more monitoring, purifying or control means, these being shown schematically as 32. These include, but are not limited to pressure gauges, flow meters, valves and filters.

The outlet line 27 allows the reaction vessel 24 to be depressurised via the outlet unit 23. The depressurised fluid can exit the sterilisation vessel 24 via line 27 and would be directed to outlet unit 23. The gas and/or fluid reaching outlet unit 23 may be separated from any additive added (separation unit not shown) and the gas can then be exhausted (exhaust line not shown). Any additive separated and optionally collected from the exhaust gas and/or fluid can be reused or disposed of as required (collection unit not shown). The outlet unit may comprise one or more monitoring systems which can be used to monitor the nature of the exhaust products as required. The SCF can also be collected, purified and/or recycled for reuse as required.

The sterilisation vessel 24 is suitably robust to withstand the temperature and pressure conditions involved in the sterilisation process. Preferably the sterilisation vessel 24 is made from stainless steel (e.g. 316 gauge stainless steel). The sterilisation vessel comprises at least one sterilisation chamber, which has a volume which is capable of accommodating the materials to be sterilised in the SCF sterilisation process, the size of the sterilisation chamber being principally dictated by use (e.g. commercial, research or laboratory scale).

The sterilisation vessel 24 may include one or more means of monitoring and/or controlling the local environment within the sterilisation vessel (not shown in FIG. 3). These may include a vibration control, temperature control and fluid agitation means. These include, but are not limited to a vibrator unit, thermostat, heater, cooler, pump, impeller and a magnetic driver.

The sterilisation vessel 24 may also be equipped with one or more internal scaffolds 33 to support the substrates to be sterilised. The scaffolds may take the form of one or more removable multi-tiered baskets, which are preferably constructed of 316 gauge stainless steel. The internal scaffold 33 is capable of supporting and protecting the items to be sterilised and can be arranged to preferentially direct the sterilant fluid in a controlled manner during agitation of the SCF in the sterilisation vessel. This may take the form of one or more directional fins, optionally with an impeller to direct flows.

The sterilisation vessel 24 may be operated at a substantially constant pressure.

Alternatively, sterilisation vessel 24 may be operated in a pressure cycling fashion. Pressure cycling involves a cyclical process of pressurisation and depressurisation. Valves within the various parts of the system allow the sterilisation vessel 24 and the other various components of the system to be isolated from each other as required. As such, the sterilisation vessel can be maintained at a substantially constant pressure, or can be periodically fully or partially depressurised and then re-pressurised again as desired.

The sterilisation vessel can be equipped with one or more access ports to allow the loading and unloading of the substrates to be sterilised. For example, the top portion of the sterilisation vessel (which may be equipped with attaching means for the various input or output lines e.g. 25, 26 and 27 and/or monitoring devices) may be readily detachable from the bottom portion of the sterilisation vessel (separable portions of vessel 24 are not shown).

Consequentially, the sterilisation vessel can be isolated from the rest of the sterilisation system, and the bottom portion of the sterilisation vessel separated from the top portion of the sterilisation vessel. In this open configuration (not shown), the substrates to be sterilised (and the one or more MVUs) can be conveniently inserted or removed from the sterilisation vessel 24.

In use, the substrate to be sterilised (not shown in FIG. 3) are placed in the sterilisation vessel 24 (e.g. in scaffolds 33). One or more MVUs are added as required. Optionally, the sterilising additive can be added at this time. If require, further additive can also be added (e.g. via line 26 from the additive source unit 22). The sterilisation vessel can then be connected to the various input or outlet lines (e.g. 25, 26 and 27).

The sterilisation vessel 24 can then be filled with the gas to be used as the SCF, the appropriate operating temperature and pressure conditions established to give a SCF or near SCF conditions (e.g. for carbon dioxide this could be between about 1000 psi to about 3500 psi, at temperatures in the range between about 25° C. to about 60° C. and for a time from about 20 minutes to about 12 hours), and the fluid agitation means activated as required to circulate the SCF in the sterilisation chamber. More preferably a time of 40 minutes to 240 minutes, most preferably 90 minutes to 140 can be used.

Further gas or additives can be added as required during the process. It is also conceived that the SCF could be prepared in a separate vessel and the SCF introduced into the sterilisation vessel 24 containing the substrate to be sterilised.

In addition, the additive line 26 may be incorporated into inlet line 25, or inlet line 25 could optionally pass through the additive source unit to direct the additive into the sterilisation vessel 24.

In order to obtain pressure cycling conditions in the sterilisation vessel 24 during operation, some of the SCF can be allowed to escape (or can be otherwise removed from) the sterilisation vessel 24 (e.g. via outlet line 23) temporarily causing a pressure drop in the sterilisation chamber. Further SCF can then be reintroduced via inlet line 25. The pressure can be allowed to drop to ambient pressure in the pressure cycling. It is also conceived that the pressure chamber of the sterilisation apparatus could be depressurised prior to condensation of the SCF. As a result of depressurisation, the SCF could more readily infuse into the substrates to be sterilised.

The sterilisation additive(s) are preferably introduced into the sterilisation vessel 24 in measured amounts, preferably in portions during the pressure cycling when the vessel is at ambient pressure. One or more additives added prior to the full pressurisation of the sterilisation vessel do not alter the final internal pressure of the pressurisation vessel. However, if the additive(s) were added when the system was fully pressurised, this would cause an increase in the internal pressure, however, this could be corrected by a control and monitoring system (not shown) as required. The mechanism for the addition of the additive(s) can be chosen and optimised as required.

Pressure cycling (i.e. depressurisation and re-pressurisation) can be repeated a number of times to best suit the sterilisation needs required. The sterilisation process can be manually operated or could be automated via a control/monitoring system (not shown in FIG. 3). For example a computer attached to appropriate sensors and control means could be used to automate the sterilisation process. The computer can be programmed to run the various sterilisation routines.

A vibrating means could be used to intermittently or continuously agitate of the reactor vessel and/or to vibrate the contents of the sterilisation chamber during the sterilisation method.

Agitation is thought to improve the mass transfer of the supercritical sterilisation fluid, thereby avoiding possible voids in the SCF. Vibration is thought to help ensure that the substrates to be sterilised are more intimately contacted with the sterilising fluid. The mechanism for vibration can be chosen and optimised as required.

Presently, the exact mechanism by which the sterilisation of substrates is achieved using SCFs (optionally with added additives) is not fully understood. It is theorised (and the invention should not be understood to be limited to this understanding) that the SCF and the chemical sterilisation additives (if employed) increase the acidity within the bacterial cell (for example by making carboxylic acid from the reaction of carbon dioxide with water). It is believed that this is especially true in the presence of water. Moreover, it is thought that suitable additives can enhance the permeability of the cell to the SCF. It is theorised that the SCF used in the sterilisation process may permanently inhibit the necessary cellular processes within the cells. It is also possible that the SCF extracts essential cellular components needed for cellular activity. SCFs may also simply be a vehicle to deliver sterilants to the targeted species to be sterilised.

It should be understood that aspects of the invention are not limited to the disclosure of the embodiments described herein, but that this disclosure is intended to cover various modifications and equivalent arrangements included within the spirit and scope of the present disclosure.

### EXAMPLES

### Example 1

### Preparation of a MVU Using (a) Soft Tissue or (b) Donor Bone as the Indicator Medium

### (i) Preparing the Indicator Medium:

The donor bone (e.g. metaphyses of long bones and whole hemipelvis) or soft tissue grafts (e.g. tendon, Achilles tendon, cartilage, ligament, skin connective or musculoskeletal tissue) is cleaned of substantially all extraneous tissue (e.g. muscle and ligament attachments).

The donor bone/soft tissue (i.e. the indicator medium), is lavaged (a washing step) with warm (approximately 40° C.) 0.9% vol sodium chloride solution until the rinsate is clear (typically a minimum of 3 litres is required).

The indicator medium is optionally divided by cutting and/or milling (depending on the intended use). When cut these pieces are about 2 to 5 cm in size. When milled (typically for Cancellous bone), the resultant granules are from 1 to 7 mm in size, or if a powder is required (typically for Cortical bone) the particles are about 50 to 900 μm in diameter. Cortical pieces can be divided as above or can be used without dividing. The granules or powders can be passed through sizing meshes to give more uniformly sized granules/powders if desired. The milled product is usually made of about 80% Cancellous bone to about 20% Cortical bone. A mixture of Cancellous and Cortical bone is known as Cortical Cancellous Crunch.

The indicator medium is then lavaged with warm (approximately 40° C.) 0.9% vol sodium chloride solution until the rinsate is clear (typically a minimum of 3 litres is required).

The indicator medium is separated into approximately even portions, typically each weighing less than 120 grams. These portions are weighed and wrap in sterile surgical gauze and each of these gauze-wrapped parcels are placed into a suitably sized Tyvek pouch (e.g. about 15×20 cm), and these are heat sealed.

### (ii) SCCD Pre-Treatment of the Indicator Medium

Approximately 32 ml of warm (approximately 40° C.) 0.9% vol sodium chloride solution is added to a double additive pad (approximately 16 ml per pad, the pad comprising a sterilisation additive e.g. NovaKill), and the treated pads placed in a pad holder which is then placed in a first stainless steel basket (approximately 1 inch high and 12 inches in diameter), and this first basket is place in the SCCD apparatus (i.e. a Nova2200 apparatus) and is position above the impellor.

The outside of the Tyvek pouches (which are loaded with the gauzed-wrapped indicator mediums) are pre-conditioned with approximately 10 ml of warm (approximately 40° C.) 0.9% vol sodium chloride solution.

The pre-conditioned Tyvek pouches are placed horizontally in a second stainless steel wire basket (approximately 7 inches high and 12 inches in diameter). The second basket is placed on top of the first basket.

A third stainless steel wire basket (approximately 5 inches high and 12 inches in diameter) containing further pre-conditioned Tyvek pouches (loaded with the gauze-wrapped indicator mediums) is prepared in the same manner as the second stainless steel wire basket. The third basket is placed on top of the second basket. All three baskets being within the SCF chamber of the SCF apparatus.

The SCF apparatus is sealed and secured, and the SCCD pre-treatment cycle is run (typically at 35° C., 9900 kPa for about 30 minutes).

When the pre-treatment cycle is complete, the SCF chamber is opened and the Tyvek-bagged indicator mediums are removed. The gauze-wrapped indicator mediums are removed from the Tyvek pouches. The gauze (containing any material extracted from the indicator medium by the SCCD) is removed from the indicator medium. The indicator medium is lavaged with warm (approximately 40° C.) 0.9% vol sodium chloride solution, until the rinsate is clear (typically a minimum of 3 litres is required).

The indicator medium is agitated in warm 40° C. 0.9% vol sodium chloride solution containing a surfactant (e.g. 5 ml of Triton-X added to 95 ml of the saline solution) for approximately 45 minutes, and the surfactant solution is then decanted off. The indicator medium is then lavaged with a warm (approximately 40° C.) 0.9% vol sodium chloride solution. The sodium chloride solution is decanted off.

### (a) When the Indicator Medium is Soft Tissue:

The indicator medium is treated with a solution of 0.3% hydrogen peroxide and 0.9% vol sodium chloride at ambient temperature (approximately 20° C.) for about 45 minutes in an ultrasonic bath.

The hydrogen peroxide solution is decanted off and the indicator medium is lavaged with warm (approximately 40° C.) 0.9% vol sodium chloride solution until the rinsate is clear. (typically a minimum of 3 litres is required).

The hydrogen peroxide treated indicator medium (about 15 grams) is placed into an appropriately size Tyvek Pouch (typically 5×7 cm) and is heat sealed.

The Tyvek Pouches loaded with the indicator medium are then placed in to an ultra low temperature freezer (at about −80° C.) until they are ready for inoculation.

### (b) When the Indicator Medium is Donor Bone:

The indicator medium is treated with 0.3% solution of hydrogen peroxide and 0.9% vol sodium chloride at ambient temperature (approximately 20° C.) for about 22 hours in an ultrasonic bath.

The hydrogen peroxide solution is decanted off and the indicator medium is lavaged with warm (approximately 40° C.) 0.9% vol sodium chloride solution until the rinsate is clear (typically a minimum of 3 litres is required).

The indicator medium is then soaked in 70% vol isopropanol (in water) for about 30 minutes, and the isopropanol solution is decanted off.

The isopropanol treated indicator medium (about 15 grams) is placed in to an appropriately size Tyvek Pouch (typically about 5×7 cm) and heat sealed.

The Tyvek Pouches loaded with the indicator medium are then placed in to an ultra low temperature freezer (at about −80° C.) until it is ready for inoculation.

(iii) Inoculating with the Indicator Medium:

The Tyvek pouches (loaded with the indicator medium), if frozen, are allowed to warm to approximately ambient temperature (approximately 20° C.). The pouches are opened and transferred to a new Tyvek pouch. The indicator medium is then inoculated with a Bacillus Atrophaeus (formerly Bacillus subtilis var Niger) spore suspension (2×10⁶ CFU) to give a sterilisation indicator.

The sterilisation indicator is heat sealed into the Tyvek pouch forming the MVU.

The MVU is placed in to a second Tyvek pouch and this second pouch is heat sealed, giving a single-bagged MVU (i.e. the sterilisation indicator housed within two gas-permeable containers). This single-bagged MVU is then placed in to a further Tyvek pouch and this further pouch is heat sealed, forming a double-bagged MVU (i.e. the sterilisation indicator is housed within three gas-permeable containers).

The double-bagged MVU is stored in an ultra low temperature freezer (at approximately −80° C.) until required. Typically the MVU is used or disposed of within 60 days.

### Example 2

### Use of MVU of Example 1

Three double-bagged MVUs are removed from the ultra low freezer and allowed to warm to ambient temperature (approximately 20° C.). The outer Tyvek bag is removed giving a single-bagged MVU.

One MVU (a positive control) is sent to quality control screening using the (e.g. British Pharmacopeia/European Pharmacopeia 2012 standards to ensure that three MVUs (made in the same batch) have the minimum number of CFUs. In this case, if a SAL of 10⁻⁶ (i.e. SAL10⁻⁶) is required of the sterilisation process a minimum of 10⁶ CFU (i.e. Bacillus Atrophaeus spores) will need to be present in the MVU.

### SCCD Sterilisation:

Approximately 30 ml of warm (approximately 40° C.) 0.9% vol sodium chloride solution is added to a double additive pad (approximately 16 ml per pad, the pad comprising the sterilisation additive e.g. NovaKill) and the treated pads placed in a pad holder which is then placed in a first stainless steel basket (approximately 1 inch high and 12 inches in diameter), and this first basket is place in to the SCCD sterilisation apparatus (i.e. a Nova2200, a 20 litre apparatus) and position above the impellor.

The outside of the two remaining MVUs are pre-conditioned with warm (approximately 40° C.) 0.9% vol sodium chloride solution. The samples to be sterilised (e.g. tissue for human transplant) are also pre-conditioned in the same way as the MVUs.

One of the pre-conditioned MVUs and a portion of the material in need of sterilisation are placed horizontally in a second stainless steel wire basket (approximately 7 inches high and 12 inches in diameter). The second basket is placed on top of the first basket.

A third stainless steel wire basket (approximately 5 inches high and 12 inches in diameter) is prepared in the same manner as the second stainless steel wire basket. The third basket containing the final MVU and the remaining portion of the material in need of sterilisation. The third basket is placed on top of the second basket. All three baskets being within the sterilisation chamber of the SCF sterilisation apparatus. Each basket containing the material in need of sterilisation also contains a MVU.

The SCCD sterilisation apparatus is sealed and secured, and the SCCD sterilisation cycle is run (typically at 35° C., 9900 kPa and impellor speed 600 rpm, for about 120 minute), followed by an appropriate purging cycle (i.e. to remove the SCCD and any materials contained therein).

When the purging cycle is complete, the sterilisation chamber is opened and the MVUs are separated from the sterilised materials.

The SCCD treated MVUs are sent for quality control testing (e.g. using the British Pharmacopeia/European Pharmacopeia 2012 standards). The sterilisation process is deemed successful if these samples are determined to be sterile. That is, where the positive control has at least 10⁶ CFUs and the SCF treated MVU are sterile; this confirms that a SAL of 10⁻⁶ (i.e. SAL10⁻⁶) was verifiably obtained in the SCCD sterilisation process, and so the resultant sterilised materials were sterilised under conditions suitable to bring about a SAL of 10⁻⁶.

The SCCD sterilised materials obtained in the SCF sterilisation process are suitably catalogued and placed in an ultra low temperature freezer (at approximately −80° C.) until required. Typically the SCCD sterilised materials obtained in the sterilisation process can be used within two years of sterilisation.

### Example 3

### SCCD Sterilised Materials

Where donor material is used, that donor material will be selected according to the requirements of the skilled person, typically meeting National legal/regulatory requirements (e.g. Standards for Tissue Banking 12^{th} Edition: American Association of Tissue Banks, incorporated herein by reference). Typically this will constitute ‘transplant grade’ donor tissue.

The following sterilised materials can be obtained in the process of Example 2, from the following sources:

### (a) Cadaverous Donor Bone (Single Source)

The transplant grade donor bone (e.g. metaphyses of long bones and/or pelvic bone) of a single human cadaver is cut into pieces of about 2 to 5 cm in size.

The donor bone (i.e. the material in need of sterilisation), is cleaned of substantially all extraneous soft tissue.

The material in need of sterilisation, is lavaged (a washing step) with warm (approximately 40° C.) 0.9% vol sodium chloride solution until the rinsate is clear (typically a minimum of 3 litres is required).

The material in need of sterilisation is divided by cutting and/or milling (depending on the intended use). When cut these pieces are about 2 to 5 cm in size. When milled (typically for Cancellous bone), the resultant granules are from 1 to 7 mm in size, or if a powder is required (typically for Cortical bone) the particles are about 50 to 900 μm in diameter. Cortical pieces can be divided as above or can be used without dividing. The granules or powders can be passed through sizing meshes to give more uniformly sized granules/powders if desired. The milled product is usually made of about 80% Cancellous bone to about 20% Cortical bone.

The material in need of sterilisation is then lavaged with warm (approximately 40° C.) 0.9% vol sodium chloride solution until the rinsate is clear (typically a minimum of 3 litres is required).

The material in need of sterilisation is separated into approximately even portions, typically each weighing less than 120 grams. These portions are placed into a suitably sized Tyvek pouch (about 8.5×12.5 cm) and heat sealed. The Tyvek pouch is then placed in a second Tyvek pouch (about 10×15 cm) and heat sealed.

The Tyvek bagged material in need of sterilisation is stored in an ultra low temperature freezer (at approximately −80° C.) until required for sterilisation. Typically the Tyvek bagged material in need of sterilisation is used or disposed of within 60 days.

### (b) Cadaverous Donor Bone (Multiple Source)

The transplant grade donor bones (e.g. metaphyses of long bones and/or pelvic bone) of at least two human cadavers are processed substantially in the same fashion as (a) above.

The material from each cadaver can be processed separately, and then mixed prior to sterilisation, or the material from each cadaver can be combined prior to the dividing step, or can be combined at any stage in between.

The Tyvek bagged material in need of sterilisation is stored in an ultra low temperature freezer (at approximately −80° C.) until required for sterilisation. Typically the Tyvek bagged material in need of sterilisation is used or disposed of within 60 days.

### (c) Live Donor Bone (Multiple Source)

The donor bones obtained from multiple living persons (e.g. femoral head tissue i.e. from hip replacement operations or metaphyses of long bones resulting from amputation) are processed substantially in the same fashion as (a) above.

The material from each live donor can be combined prior to the dividing step, or can be combined at any stage in between.

The Tyvek bagged material in need of sterilisation is stored in an ultra low temperature freezer (at approximately −80° C.) until required for sterilisation. Typically the Tyvek bagged material in need of sterilisation is used or disposed of within 60 days.

### (d) Demineralised Bone Matrix

Demineralised Bone Matrix (DBM) is a well-known material first discovered in 1965 (Urist, Science, (1965), 150:893-899, incorporated herein by reference).

DBM can be prepared in a number of ways, these methods being known to the skilled person (e.g. Urist, Science, (1965), 150:893-899, US 2009/0226523 (Dorsey & Whitney LLP) and the references cited therein, these being incorporated herein by reference).

DBM is bone in which inorganic minerals have been removed, leaving behind an organic collagen matrix. As a result of the demineralization process, DBM is more biologically active than bone that has not been demineralised.

That is, DBM is derived from donated human cadaver bone that is ground and demineralised using a series of acid baths and includes certain grinding and drying steps.

Bone is about 70% mineral by weight. The remaining 30% is collagen and non-collagenous proteins (including Bone Morphogenic Proteins—BMP's). Making DBM exposes the natural BMP's, so bone growth and remodelling can occur.

The DBM matrix having particles which are about 50 to 900 μm in diameter is prepared using standard methods known to the skilled person. This powder can be passed through sizing meshes to give more uniformly sized powders if desired.

The demineralised bone or the demineralised bone matrix once prepared is placed into a suitably sized Tyvek pouch (about 8.5×12.5 cm) and heat sealed. The Tyvek pouch is then placed in a second Tyvek pouch (about 10×15 cm) and heat sealed.

The Tyvek bagged material in need of sterilisation is stored in an ultra low temperature freezer (at approximately −80° C.) until required for sterilisation. Typically the Tyvek bagged material in need of sterilisation is used or disposed of within 60 days.

### (e) DBM Paste

The DBM is prepared as in (d) above, however, prior to placing in a Tyvek container, the DBM is mixed with a suitable carrier (i.e. osteoconductive carrier) to form a paste. The paste formed can be used by orthopaedic surgeons for bone repair and bone regeneration.

Suitable carriers for the paste are known to the skilled person and include silicone, gels or liquid polymer or a mixture of these. The ratio of bone to carrier will depend on the consistency of the paste desired, for example a stiff paste may contain 80% bone to 20% carrier, whereas a more mobile paste may contain only 20% bone. Typically 1 to 25 grams of DBM will be used in the paste.

The paste formed can be packed into a gas-permeable squeezable tube (e.g. about 5 cm long and 1 cm wide to 20 cm long and 3 cm wide) and the tube sealed. The tube may take a syringe-like form, having a barrel and plunger. The seal may also include a removable cap. The sealed tube is placed in to a suitably sized Tyvek pouch and heat sealed. Optionally, the Tyvek bagged tube is then placed in to a second Tyvek pouch and heat sealed.

The Tyvek bagged material in need of sterilisation is stored in an ultra low temperature freezer (at approximately −80° C.) until required for sterilisation. Typically the Tyvek bagged material in need of sterilisation is used or disposed of within 60 days.

### (f) Soft Tissue

The transplant grade donor soft tissue (e.g. tendon, Achilles tendon, cartilage, ligament, skin or connective tissue or musculoskeletal tissue), normally of a single human cadaver is cut into pieces if desired, of about 1 to 5 cm in size.

The donor soft tissue (i.e. the material in need of sterilisation), is cleaned of substantially all extraneous tissue.

The material in need of sterilisation is lavaged with warm (approximately 40° C.) 0.9% vol sodium chloride solution until the rinsate is clear (typically a minimum of 3 litres is required).

The material in need of sterilisation is separated into approximately even portions, typically each weighing less than 120 grams. These portions are placed into a suitably sized Tyvek pouch (about 16×26 cm) and heat sealed. The Tyvek pouch is then placed in a second Tyvek pouch (about 18×28 cm) and heat sealed.

The Tyvek bagged material in need of sterilisation is stored in an ultra low temperature freezer (at approximately −80° C.) until required for sterilisation. Typically the Tyvek bagged material in need of sterilisation is used or disposed of within 60 days.

### (g) Non-Biological Tissue

The material in need of sterilisation would be provided in the form ready for the terminal SCCD sterilisation, and may be in one or more parts. It might also be pre-treated with a SCF to remove any materials that are extractable into the SCF (like grease).

The material in need of sterilisation is placed into a suitably sized Tyvek pouch and heat sealed. The Tyvek pouch is then placed in a second Tyvek pouch and heat sealed. The Tyvek bagged material is stored under suitable conditions until required for sterilisation and used or disposed of within 60 days.

1. A Method Validation Unit (MVU) for the evaluation of the level of sterilisation in a sterilisation method involving the use of a fluid at or near the supercritical pressure and temperature for that fluid, wherein the MVU comprises:
a sterilisation indicator housed within a gas-permeable container, wherein the sterilisation indicator comprises an indicator medium and a population of one or more colony forming units (CFUs), and wherein the indicator medium comprises one or more structural features representative of the internal structure of a material to be sterilised in the sterilisation method.

2. A MVU according to claim 1 wherein the population of the one or more CFUs is at least 10³ CFUs, 10⁶ CFUs or 10¹² CFUs.

3. A MVU according to claim 1 or 2, wherein the population of the one or more CFUs is 2×10⁶ CFUs.

4. A MVU according to anyone of claims 1 to 3, wherein the indicator medium comprises a porous structure.

5. A MVU according to anyone of claims 1 to 4 wherein the gas-permeable container is substantially gas-permeable over its entire surface.

6. A MVU according to anyone of claims 1 to 5 wherein the container comprises a non-woven polymeric material.

7. A MVU of any one of claims 1 to 6, wherein the MVU is housed within a second gas-permeable container.

8. A MVU according to any one of claims 1 to 7 wherein the indicator medium comprises one or more materials selected from: tissue suitable for transplant, research or therapeutical grade bone, demineralised bone, demineralised bone matrix, a paste comprising demineralised bone or demineralised bone matrix, whole cortical bone pieces, tendon, Achilles tendon, cartilage, ligament, skin, connective or musculoskeletal tissue or biological tissue suitable for implantation; natural or synthetic polymer, biomedical polymer, medical-grade polymer, biodegradable polymer, synthetic human tissue analogue; biologically active molecules, pharmaceutically active compounds, pharmaceutical carriers, or pharmaceutical delivery vehicles; metals, alloys, medical equipment, instruments, prosthetics, implants or representative analogues thereof.

9. A MVU according to any one of claims 1 to 8 wherein the indicator medium comprises Cortical Cancellous Crunch.

10. A MVU according to anyone of claims 1 to 9 wherein the indicator medium comprises human-derived tissue.

11. A MVU according to claim 8 wherein the synthetic tissue analogue comprises silica, glass or a ceramic material.

12. A MVU of any one of claims 1 to 11 wherein the indicator medium is granular, powder or fibrous in nature and/or formed into a solid mass.

13. A MVU of any one of claims 1 to 12 wherein the indicator medium comprises granules of between about 1 and 9 mm, 2 and 7 mm, or 3 and 5 mm, and where the indicator medium comprises a powder, the powder has a mean particle diameter of about 10 to 900 μm, 100 to 700 μm, or 150 to 350 μm.

14. A MVU according to anyone of claims 1 to 13 wherein the one or more CFUs are selected from bacteria, vegetative microbial cells, moulds, single-celled organisms, protozoa, yeasts, viruses, or other infective agents spores, or progenitor species thereof.

15. A MVU according to anyone of claims 1 to 14 wherein the one or more CFUs are selected from B. Atrophaeus (formerly B. subtilis var Niger), B. stearothermophilus, B. subtilis, B. pumilus, B. cereus, Listeria innocua, Staphylococcus aureus, Salmonella salford, Psuedomonas aeruginosa, Escherichia coli, Preoteus vulgaris, Legionella dunnifii; spores, or progenitor species thereof, or wherein the one or more CFUs is a 2×10⁶ B. Atrophaeus spore suspension.

16. A MVU according to any one of claims 1 to 15, wherein the indicator medium has been pre-treated with a SCF, or wherein the indicator medium has been pre-treated with a SCF and wherein that SCF was SCCD.

17. A MVU according to claim 16 wherein the pre-treatment has removed 25 to 99% of the mass of the material pre-treated, or has removed 70 to 98% of the mass of the material pre-treated, or has removed 85 to 95% of the mass of the material pre-treated.

18. A blister pack comprising two or more MVUs according to anyone of claims 1 to 17, wherein the MVUs are detachable from the blister pack.

19. A sealable gas-permeable container for use in a sterilisation method, wherein the sealable container comprises a MVU according to anyone of claims 1 to 17.

20. A MVU kit for use in the evaluation of a sterilisation method, the kit comprising:
a sealable gas-permeable container, a sterilisation indicator medium, and a carrier medium, wherein the indicator medium comprises one or more structural features representative of the internal structure of the material to be sterilised in the sterilisation method, and wherein the carrier medium comprises a population of one or more CFUs, wherein the indicator medium and the carrier medium are combinable to form a sterilisation indicator.

21. A method of manufacturing a MVU according to anyone of claims 1 to 17 comprising the step of housing the sterilisation indicator in the gas-permeable container, wherein the sterilisation indicator comprises an indicator medium and a population of one or more CFUs, and wherein the indicator medium comprises one or more structural features representative of the internal structure of the material, to be sterilised in the sterilisation method, and wherein the MVU is useful in evaluating the level of the sterilisation in the sterilisation method.

22. A method of manufacturing a sterilisation indicator as defined in anyone of claims 1 to 17 comprising the steps of: (i) optionally substantially sterilising the indicator medium; (ii) optionally pre-treating the indicator medium with a SCF; (iii) treating the indicator medium with a carrier medium, the carrier medium comprising a population of one or more CFUs, (iv) optionally incubating the treated indicator medium.

23. A method of manufacturing according to claim 22, wherein the indicator medium is pre-treated with a SCF, or wherein the indicator medium is pre-treated with a SCF and wherein the SCF is SCCD.

24. A method of manufacturing according to any one of claim 22 or 23 wherein the pre-treatment removes 25 to 99% of the mass of the material pre-treated, or removes 70 to 98% of the mass of the material pre-treated, or removes 85 to 95% of the mass of the material pre-treated.

25. Use of a MVU according to any one of claims 1 to 18 in an apparatus for sterilisation, wherein the sterilisation method comprises the use of a fluid at or near the supercritical pressure and temperature for that fluid.

26. A sterilisation method comprising bringing a MVU and a material in need of sterilisation into contact with a sterilant fluid, the sterilant fluid comprising a fluid at or near the supercritical pressure and temperature for that fluid, and wherein the MVU is useful in evaluating the level of the sterilisation in the sterilisation method.

27. A sterilisation method according to claim 26 wherein the MVU is a MVU according to any one of claims 1 to 18.

28. A sterilisation method according to any one of claim 26 or 27 wherein the sterilisation method is a terminal sterilisation method.

29. A sterilisation method according to claims 26 to 28 wherein the fluid is carbon dioxide.

30. A sterilisation method according to any one of claims 26 to 29 wherein the sterilant fluid comprises one or more additives selected from: a peroxide, carboxylic acid, acid anhydride, ester or alcohol, or is a commercial additive package.

31. A sterilisation method according to any one of claims 26 to 30 wherein the additive is present in an amount of between about 0.001% to about 2.0% based on the total volume of the sterilant fluid.

32. A sterilisation method according to any one of claims 26 to 31 wherein the material in need of sterilisation is from a human donor.

33. A sterilisation method according to claim 32 wherein the material in need of sterilisation is from one or more donors.

34. A sterilisation method according to any one of claim 32 or 33 wherein the material in need of sterilisation is from one or more live donors or from one or more cadaveric donors or is a mixture of live and cadaveric donors.

35. A sterilisation method according to any one of claims 26 to 34 wherein the sterilisation chamber is 20 to 200 litres, 60 to 150 litres, or 80 to 100 litres.

36. A sterilisation method according to any one of claims 26 to 35, wherein the material in need of sterilisation has been pre-treated with a SCF, or wherein the material in need of sterilisation has been pre-treated with a SCF and wherein that SCF was SCCD.

37. A sterilisation method according to claim 36 wherein the pre-treatment has removed 25 to 99% of the mass of the material pre-treated, or has removed 70 to 98% of the mass of the material pre-treated, or has removed 85 to 95% of the mass of the material pre-treated.

38. A sterilised material obtained by the sterilisation method of anyone of claims 26 to 37, or article derived thereof.

39. A sterilised material obtained by the sterilisation method according to any one of claims 26 to 38, or article derived thereof, wherein the sterilised material has been sterilised under conditions sufficient to achieve at least a 6-log or 12-log reduction, in a population of one or more CFUs.

40. A sterilised material obtainable by the sterilisation method according to anyone of claims 26 to 37, or article derived thereof, wherein the sterilised material has been sterilised under conditions sufficient to achieve at least a 6-log or 12-log reduction, in the population of one or more CFUs, and wherein this reduction is verifiable.

41. A sterilised material according to anyone of claims 38 to 40, or article derived thereof, wherein the material is selected from therapeutic grade tissue suitable for transplant, bone, demineralised bone, demineralised bone matrix, a paste comprising demineralised bone or demineralised bone matrix, whole cortical bone pieces, tendon, Achilles tendon, cartilage, ligament, skin, connective or musculoskeletal tissue or biological tissue suitable for implantation; natural or synthetic polymer, biomedical polymer, biodegradable polymer, synthetic human tissue analogue; biologically active molecules, pharmaceutically active compounds, pharmaceutical carriers, or pharmaceutical delivery vehicles; metals, alloys, medical equipment, instruments, prosthetics or implants.

42. A sterilised material according to anyone of claims 38 to 41, or article derived thereof, wherein the material comprises Cortical Cancellous Crunch.

43. A sterilised material according to anyone of claims 38 to 42, or article derived thereof, wherein the article derived thereof is selected from an allograft, implant, stent, catheter, endoscope, prosthesis, joint replacement, medical scaffolding, suture, medical instrument, surgical instrument, drug delivery device or pharmaceutically active implant or microparticles.

44. Use of a sterilised material, or article derived thereof, according to anyone of claims 38 to 43 in a method of medical treatment.

45. A method of medical treatment comprising the use of a sterilised material, or article derived thereof, according to anyone of claims 38 to 43.

46. Use of a sterilised material, or article derived thereof, according to anyone of claims 38 to 43 in the manufacture of a product for use in implantation or transplantation.

47. A pre-treatment method, wherein a material in need of sterilisation is pre-treated with a SCF, or wherein the material in need of sterilisation is pre-treated with a SCF and wherein that SCF is SCCD.

48. A pre-treated material obtained or obtainable by the pre-treatment method of claim 47.

49. A pre-treated material according to claim 48, wherein the pre-treatment method has removed 25 to 99% of the mass of the material pre-treated, or has removed 70 to 98% of the mass of the material pre-treated, or has removed 85 to 95% of the mass of the material pre-treated.

50. A sterilisation method comprising bringing the pre-treated material of any one of claim 48 or 49 in need of sterilisation into contact with a sterilant fluid, wherein optionally the sterilant fluid comprises a fluid at or near the supercritical pressure and temperature for that fluid.
